(19) United States
(12) Patent Application Publication
(10) Pub. No.: US 2024/0036034 A1
(43) Pub. Date: Feb. 1, 2024

(54) METHOD FOR SCREENING OF A CHEMICAL SUBSTANCE

(71) Applicants: Bayer Aktiengesellschaft, Leverkuse (DE); Targenomix GmbH, Potsdam (DE)

(72) Inventors: Sebastian KLIE, Potsdam (DE); Florian SCHRÖDER, Berlin (DE); Marco BUSCH, Niedernhausen-oberjosbach (DE)

(21) Appl. No.: 18/266,207

(22) PCT Filed: Nov. 23, 2021
(86) PCT No.: PCT/EP2021/082701

§ 371 Date: Jun. 8, 2023

(30) Foreign Application Priority Data
Dec. 17, 2020 (EP) 20215187.4

(51) Int. Cl. G01N 33/50

(52) U.S. Cl. CPC G01N 33/5097; G01N 2333/415

(57) ABSTRACT

The invention relates to a method for screening of at least one chemical substance by treatment of plant material, comprising the following process steps: a) Applying the plant material into a cavity; b) Treatment of the plant material with the chemical substance; c) Creating at least one dataset showing at least one phenotypical characteristic of the plant material after treatment with the chemical substance; and d) Assigning the chemical substance based on the dataset to at least one site of action (SoA) and/or at least one mode of action (MoA) of a multitude of stored SoA and/or MoA by using a SoA- and/or MoA-compendium containing data regarding dependencies between phenotypical characteristics of at least one plant material treated by at least one reference substance of a known SoA and/or MoA.

The present invention relates to a method for screening of at least one chemical substance for a treatment of plant material.

Intensive screening of chemical substances concerning their impact on plant material is commonly conducted during the development process of chemical substances used for plant treatment. A profound knowledge of how a specific chemical substance affects plants is essential for successful treatment of plants. In case the chemical substance is a pesticide, for example an herbicide, fungicide or insecticide, it is very important to avoid the emergence of resistances: different chemical substances may share a same site of action (SoA) and/or mode of action (MoA) on plant material. Repeatedly using the same chemical substance or different chemical substances that share the same SoA and/or MoA, for example in form of herbicides, may lead to resistances. To avoid this, the chemical substances should preferably be intensively examined before they enter the market.

Evaluation of chemical substances used for agricultural plants are often carried out under field conditions or in greenhouses, as for example shown in EP 1 777 486 B1. This kind of screening has several disadvantages as for example the predominance of inconsistent climate conditions between different trials. Differences in temperature or rainfall may affect the outcome of a field investigation, which, in consequence, worsens comparability of data. Another disadvantage of investigations in field environments or in greenhouses is the vast amount of space required and the time-consuming cultivation of the plants. Both factors lead to considerable costs for these types of evaluation of chemical substances. Further disturbance variables as, e.g., damage caused by animals or exposure to plant pathogens (from which it is difficult to protect plants under these circumstances) impair the collection of accurate data suitable for further statistical processing.

Compared to investigations on field or greenhouse level, laboratory screening is less time-consuming and provides standardized growth conditions. Several methods for quantitative, semi-automated phenotype analyses exist, as for example an imaging system as described in WO 2019/152424 A1. However, this imaging system lacks precise and high-throughput data acquisition, as well as uniform and standardized data analysis, which leads to variation within the obtained data. Consequently, these methods are not suitable for precise evaluation of a high number of applied chemical substances.

WO 2010/026218 A1 shows a method for a destructive screening of a chemical substance for a treatment of plant material. In this method the plant material is homogenized and solubilized to make it accessible for in situ recording infrared spectra (IR, FT-IR, Raman, FT-Raman and Near Infrared (NIR)). Samples prepared in this manner cannot be used to obtain specific results on individual plants, parts (organs) of a plant, or, to measure the influence of a substance on the same plant material over a period of time.

It is therefore an object of the present invention to provide a high-throughput method, which enables rapid, cost-effective, consistent and reproducible screening of a chemical substance under laboratory conditions.

This object is achieved by means of the method according to claim 1. Advantageous embodiments and developments are objects of the dependent claims.

The present invention provides a method for screening of at least one chemical substance by treatment of plant material. Plant material in this context may comprise parts of a plant (as for example roots or leaves), or, preferably, a whole plant. The growth stadium of the whole plant may comprise all stages of plant life cycle, as for example an ungerminated or germinated seed, the seedling stadium, but also different developmental or reproduction stages of a plant. The plant material may belong to a monocotyledonous or a dicotyledonous plant species (monocot and dicot).

The method according to the invention comprises several different steps, which are preferably performed one after another:

In a first step (step a), the plant material is applied into a cavity. The cavity is, in this context, defined as a surface containing a recess. However, it is also possible to use a plate without any recess for this purpose, for example an object carrier commonly used in microscopy.

In a preferred embodiment, the plant material is put on a substrate, which has multiple beneficial properties: the substrate inside the cavity can fixate and/or preserve the plant material by stabilizing its architecture and/or deliver nutrient content. This way, the plant material is put in an environment able to develop and grow in cases where the plant material is still capable of growth. The substrate may be of solid or liquid nature. In another embodiment of the invention the cavity is used without any substrate.

The plant material is preferably grown under standardized growth conditions to ensure reproducibility of phenotypic responses and achieve the desired throughput. Additional growth conditions serve to enhance the phenotypic response of substances with particular SoA and/or MoA, which effects the plant material marginally or not at all under standard growth conditions. For example, plant material can be exposed to different stressors as a further parameter of the growth regimen. Plant material grown under standard, i.e., “non-stress” growth condition may show a different phenotypical development after treatment with a specific chemical substance than plant material exposed to, e.g., abiotic stress conditions. The inclusion of different growth conditions and stressors can be helpful for reliably assigning the chemical substance to one or more SoA and/or MoA, as well as for developing recommendations for proper use of the chemical substances on a larger scale under field or greenhouse conditions. A highly standardized pre-growth of the plant material is not obligatory for the conduction of the method according to the invention, but it is advantageous to obtain a proper comparability of the datasets. In other embodiments it is also possible to use readily pre-grown plants, parts of the plant or ungerminated seeds.

In a second step (step b), the plant material is treated with the chemical substance. Different types of application may be used to treat the plant material. One possible application is pipetting of the chemical substance on top of the growth media, another possibility is foliar application by aerosol spraying the chemical substance in the cavity containing the plant material. The type of application can be selected according to the chemical substance to be analyzed or by specific target location: sprayed chemical substances may be absorbed preferentially by aerial areas of the plant, as for example by embryonic or true leaves, while pipetted chemical substances are absorbed mainly by the plant's root system via the growth medium. Application type may be adapted depending on formulation of the chemical substance optimized for plant material types, for example for monocots and dicots. In another embodiment, the chemical substance can be already present in the cavity when the plant material is applied into it.

In a third step (step c), at least one dataset capturing at least one phenotypical characteristic of the plant material after treatment with the chemical substance is created. This dataset is the base for the further investigation of the chemical substance, as it captures the effects of the chemical substance on the plant material.

In a fourth step (step d), the chemical substance is, based on the dataset created in step c, assigned to at least one SoA and/or at least one MoA of a multitude of stored SoA and/or MoA by using a previously obtained SoA- and/or MoA-compendium containing datasets regarding dependencies between phenotypical characteristics of at least one plant material treated by at least one reference substance of a known SoA and/or MoA. The compendium, which is a concise collection of information, serves as a reference for matching and assigning the phenotypic characteristics induced by a screened chemical substance to specific SoA and/or MoA: if the dataset of the screened chemical substance matches one or more datasets stored in the compendium, this specific chemical substance can then be assigned to the same SoA and/or MoA (SoA and/or MoA classification).

The MoA is in this context defined as the effect of the chemical substance on the plant material. Each single interaction of the chemical substance with any molecular target of the plant material is subsumed under this term, leading to altered or disturbed physiological processes, starting from absorption and ending with the plant material's response on the chemical substance. The SoA describes in particular, the specific biochemical interaction through which the applied chemical substance manifests its phenotypical effect by any biological means.

This can be, but is not limited to, the modulation of protein activity and/or cellular processes and/or structures. One MoA can be caused by one or more SoA. For example, one MoA affecting plant growth is very long chain fatty acid (VLCFA) biosynthesis. Multiple SoA responsible for this MoA exist and can be for example a VLCFA-synthase or VLCFA-elongase inhibitor. Both exemplified SoA lead to the same MoA but affect different synthesis processes within the plant material.

For multiple agronomically relevant herbicides, several resistant weed species have emerged until today. By identifying SoA and/or MoA of chemical substances, those chemical substances can be classified and at the same time avoidance strategies for the prevention of resistances can be developed: a chemical substance that addresses a novel SoA and/or MoA has the potential to overcome already existing resistances in the field.

According to another embodiment of the invention, the dataset is obtained by use of a sensor unit. The sensor unit is used to obtain datasets regarding phenotypical characteristics of the plant material.

According to another embodiment the sensor unit is used to obtain the dataset in a non-destructive and non-intrusive manner.

According to another embodiment of the invention, the sensor unit comprises at least one of the following digital sensors: hyperspectral VIS (visible), hyperspectral NIR (near-infrared), hyperspectral UV (ultra-violet), chlorophyll fluorescence and RGB sensor. The sensor unit may also comprise a combination of the aforementioned sensors or additional other sensors. Hyperspectral imaging sensors are particularly useful to monitor changes in molecular composition of the plant material (e.g., water content, cell density, pigment composition) and in the visible or ultra-violet, or, (near) infrared spectrum of the light and, therefore be indicative for a SoA and/or MoA.

The sensor unit may comprise a hyperspectral VIS camera to obtain imaging data of the visible light spectrum. This light spectrum can, e.g., be helpful to detect changes in pigment content of plant material induced by the application of the chemical substance.

The sensor unit may comprise a hyperspectral NIR camera to obtain imaging data of the near-infrared light spectrum. This light spectrum can, e.g., be helpful to detect changes in water content of plant material induced by the chemical substance.

The sensor unit may comprise a hyperspectral UV camera for the imaging of UV spectra. Images taken by use of this sensor can help to monitor changes in molecular composition of metabolites absorbing in the UV spectrum e.g., amino acids in the plant material.

The sensor unit may comprise a chlorophyll fluorescence measurement system, which helps to sense plant stress and to capture effects on the photosystem of the plant material. The chlorophyll fluorescence measurement system may for example comprise a camera and a high-density LED panel that emits light suitable to drive the photosynthetic reaction under which the cavity is placed.

The sensor unit may comprise a RGB camera, which captures digital photographs of the plant material e.g., to perform a color class analysis of the plant material and/or monitor growth.

According to another embodiment of the invention, a light source with a circular arrangement of lamps and reflective surface is used for the hyperspectral VIS and hyperspectral NIR sensors to homogeneously illuminate the plant material. In a preferred embodiment, the light source comprises six circular arranged halogen lamps and a highly reflective roughened aluminum surface which reflects the light directly, and through a hole in the middle to the multi-well plate (through which the sensor unit captures measurements of the plant material). This ensures homogeneously illumination of the plant material while obtaining datasets via imaging with hyperspectral VIS and hyperspectral NIR sensors. Homogenous illumination of the plant material helps to generate high-quality images and avoid errors caused by uneven illumination. High quality of the datasets facilitates further processing and increases the precision of the evaluation of the datasets.

According to another embodiment of the invention, the cavity is a well of a multi-well plate. A multi-well plate is a plate containing several wells for use under laboratory circumstances. In a preferred embodiment, the multi-well plate comprises 96 single wells. This kind of multi-well plates are commonly used in laboratories. 12 single wells are lined up in a row, and 8 columns are present on the 96 well plate, leading to a defined spatial arrangement further simplifying a sub-division of the dataset obtained of the multi-well plate into individual datasets for each well, respectively plant material inside each well. The use of 96 well plates offer a good balance between size of plant material, which relates to the amount of information, i.e., picture elements or pixels, recorded for digital data acquisition, and time required for plant growth relating to the achievable throughput for this method. It is also considered possible to use other multi-well plates, e.g., standardized multi-well plates according to the Society for Biomolecular Sciences (SBS). In a preferred embodiment, the method is carried out by use of an apparatus comprising a robot-arm used to carry the multi-well plates to the sensor unit, where datasets are obtained.

According to another embodiment, each well contains one piece of plant material. This is advantageous to ensure correct data acquisition: phenotypic data and derived parameters (as for example growth) of one piece of plant material can be extracted accurately. Data recording is simplified, if it is ensured that each well only contains one piece of plant material.

According to another embodiment of the invention, the chemical substance is applied at different concentrations. In a preferred embodiment of the invention, the effective concentration has been determined prior to classification of the chemical substance. This can be achieved for example by applying a brought concentration range and investigating the phenotypic effects, for instance an EC₅₀ value relating to effects on plant growth or photosynthetical activity on the plant material with the described imaging sensors. At least one effective concentration is selected for classification. This is advantageous for increasing the accuracy of the classification method. It can also be advantageous for later recommendations for use of a specific chemical substance at practical application in order to avoid both under- and overdosing.

According to another embodiment of the invention, one collective dataset is taken for a multitude of spatially separated plant materials, i.e., pieces, which are subsequently decomposed into single datasets per single piece of plant material. In an exemplary embodiment, one collective dataset is taken for 96 pieces of plant material at once and later decomposed into 96 single datasets, each containing data of one piece of plant material. This way, the method is suitable for high-throughput screening: the screening takes less time, as it is easier to obtain one big dataset of a higher number of plant material and later decompose it into several smaller datasets per single plant material.

According to another embodiment of the invention, multiple datasets of the plant material are obtained after treatment at different predefined times and/or by use of different sensors. In one embodiment, multiple datasets are obtained of the same plant material after treatment with a time interval of 1 h to 48 h, preferably 12 h to 30 h and particularly preferably 24 h in between the obtaining of one dataset. The starting point of obtaining datasets after treatment may be varied as well, depending on plant material, growth conditions and chemical substance used. For a time interval of 24 h between acquiring datasets, data may be obtained at, for example, 24, 48, 72 and 96 h after treatment. In an embodiment, a total number of 2 to 10 datasets (time points) per piece of plant material is obtained, preferably 3 to 7 and particularly preferred 4. These datasets help to assign the SoA and/or MoA with a higher accuracy, as some phenotypical effects of the chemical substance on the plant material evolve over time, whereas others display rapid effects: The datasets of different plant material treated by chemical substances with different SoA and/or MoA may look similar 96 h after treatment, but differ in earlier time points after treatment. It might as well occur that datasets taken at earlier time points may look the same for different SoA and/or MoA and show particular differences at a later time points. The time course can therefore be an important criterion for differentiating the individual SoA and/or MoA.

Multiple datasets can be obtained not only after different pre-defined times, but as well by the use of different sensors. In a preferred embodiment, hyperspectral imaging via VIS and NIR sensors are combined with chlorophyll fluorescence and RGB imaging. It is preferred to obtain multiple datasets by use of different sensors and after different pre-defined times. This way, the accuracy of the assignment of a chemical substance to at least one SoA and/or MoA is maximized. One advantage of the method according to the present invention is the ability to process a large number of datasets in a short time, so that large-scale data collection is feasible without problems.

According to another embodiment of the invention, a dataset of a plant material can subsequently be decomposed into single datasets of different parts of a plant material. This is advantageous especially for in-depth analysis of the effects of the chemical substance on the plant material and can facilitate the assignment to a SoA and/or MoA. For example, it is possible to determine whether the effect is particularly noticeable on the cotyledons, in the root area or on the entire plant.

According to another embodiment, an automated quantitative image analysis process is carried out via a special program on the recorded datasets, that separates regions (i.e., a set of pixels) corresponding to the plant material from those belonging to the background. In another embodiment, where a dataset containing a light spectrum is recorded, this program will extract which set of pixels corresponds to the plant material over all recorded light spectrum wavelengths. The program then corrects the obtained data and transforms it into a data-matrix complementing the SoA- and/or MoA-compendium.

According to another embodiment of the invention, at least one dataset showing at least one phenotypical characteristic of the plant material before treatment with the chemical substance is obtained. Taking a dataset of phenotypical characteristics of the plant before treatment helps to increase accuracy of the assignment to specific SoA and/or MoA: a comparison of the dataset taken before treatment with one or more datasets taken after treatment clearly shows the impact of the chemical substance on the plant. The images taken before treatment should be obtained by use of the same sensors used for taking images after treatment in order to get comparable datasets. The data should be acquired immediately before treatment for each plant material to ensure comparability.

It is also possible to compare datasets after treatment with datasets of a control group. The control group is built up by one or more pieces of plant material prepared in the same manner as the plant material to be treated, except that it is not treated with the chemical substance that is screened. Preferably, each multi-well plate contains several controls in order to identify errors in the method and to correct data.

According to another preferred embodiment of the invention, assigning of the chemical substance to at least one SoA and/or at least one MoA is carried out by using an adapted program performing a machine learning process. In a preferred embodiment, the machine learning process is supervised. It is preferably trained on a corpus of datasets of well characterized chemical substances of which their respective SoA and/or MoA are known to establish a SoA- and/or MoA-compendium.

According to another embodiment of the invention, the SoA and/or MoA compendium is augmented by recording of data of at least one reference substance of a further SoA and/or MoA. This way, created datasets for chemical substances that are assigned to a known SoA and/or MoA are saved to update the SoA- and/or MoA-compendium by inclusion of the aforementioned dataset. Advantageously, all data recorded contributes to a better understanding of different SoA and/or MoA and the phenotypical characteristics linked to them. With an increasing SoA- and/or MoA-compendium, the assignment of recorded datasets to SoA and/or MoA becomes more accurate. This way, every single recorded dataset contributes to an increased reliability of the screening method. It is preferred to use different chemical substances for the same SoA and/or MoA in order to obtain a reliable SoA- and/or MoA-compendium. Therefore, it is advantageous to record and save as many datasets as possible in the SoA- and/or MoA-compendium and to keep it steadily growing. In another preferred embodiment, only datasets for chemical substances that are assigned to a known SoA and/or MoA with a desired prediction accuracy of preferably >80% are used to update the SoA- and/or MoA-compendium.

In a preferred embodiment, the compendium contains data of at least fifteen different known SoA and/or MoA. Supervised machine learning techniques (both classical machine learning and artificial intelligence, i.e., AI) are used to build a classification model, representing a machine learning process, which is preferably able to predict the SoA and/or MoA of unknown chemical substances with a desired accuracy of preferably >50%, particularly preferable with a desired accuracy of >80%.

In another embodiment, support-vector machines (SVMs) are used as supervised learning models for the machine learning process for the classification of the SoA and/or MoA of an applied chemical substance. Associated learning algorithms of the SVM are utilized in the first step of building the SoA- and/or MoA-compendium as the training data. In another embodiment, SVMs are used as supervised learning models with associated learning algorithms that analyze the SoA- and/or MoA-compendium as the training data used for classification of the SoA and/or MoA of an applied substance.

In another embodiment, features (instead of the raw acquired datasets), which are representative for the whole recorded dataset, are extracted and used to carry out the machine learning process. These features will be selected in the machine learning process automatically and capture the phenotypical characteristics as good as or better than the primary datasets recorded.

According to another embodiment of the invention, an uncharacterized SoA and/or MoA is identified for each chemical substance, which is not assignable to any recorded SoA and/or MoA in the SoA and/or MoA compendium. This feature helps to increase the data compendium by addition of uncharacterized and/or novel SoA and/or MoA information and enhances the understanding of chemical substances and the intercellular processes they induce. This way, the method is not only suitable for assignment of chemical substances to known SoA and/or MoA, but as well for the identification of so far uncharacterized SoA and/or MoA by the described method.

According to another embodiment, the plant material is in a seed or seedling stage at step a) of the method according to the invention. This way, stratification can be applied on all plant materials and all of the plant materials grow to the exact same stage, when the chemical substance is applied to the plant material. The starting plant material should be as uniform as possible to ensure the reliability of the results. The use of plant material in a seed or seedling state is preferred in this method, as the growth to this stage takes only a short amount of time compared to later plant growth stages. Consequently, the method is suitable for high-throughput screening on laboratory scale. Time-consuming cultivation of plant materials in a later growth stage is not needed. Another advantage of the use of plant material in a seed or seedling stage is the saving of space, which makes it ideal for cost effective screening. A high number of plant material can be kept in growth chambers instead of space consuming pot or field cultivation of older and bigger plant materials. Several multi-plate wells can be kept on different levels of one or more growth chambers, whereby the growth conditions are the same for all multi-well plates and, in consequence, for all the plant material. Another advantage of the use of plant material in such an early stage is the high similarity of seedlings in that stadium. The older the plant materials become, the further the development of each single plant material drifts apart. For reasons of comparability, it is therefore advantageous to use plant material in an early stage of growth and/or development.

According to another embodiment, the plant material belongs to the plant species Arabidopsis thaliana, which has the advantages of being a representative weed with short generation cycles, well established growth conditions and properties to transfer results to other higher plant species. A fast-growing plant species has the advantage of reduced screening time and established growth conditions, which is particular advantageous for laboratory investigations with a high throughput. However, it is also possible to use other plant species for the screening, such as, but not limited to Poa annua, Matricaria chamomilla or plants belonging to the genus of Lemna.

According to another embodiment, the chemical substance is a plant growth regulator. According to the invention, plant growth regulators are chemical substances with positive or negative effects on plant growth and development. These chemical substances can belong, but are not limited to, one of the following classes: (1) phytotoxins that are either chemically synthetized small molecules or naturally occurring chemical substances, such as herbicides; (2) chemical substances that promote plant growth and/or increase yield, such as phytohormones and/or fertilizers; and, (3) chemical substances that enhance biotic and abiotic stress tolerance of plants, such as safeners that protect plants against herbicides or compounds that modulate drought tolerance. The screening of any of the aforementioned types of plant growth regulators by use of the method according to the present invention is advantageous, as they can be assigned to their SoA and/or MoA accurately and in case of novel SoA and/or MoA, a new class can be set up.

### Example 1—Plant Growth Procedure

Plant material grown under highly controlled environmental conditions is the basis to analyze effects on the plant material arising from treatment with a chemical substance. A miniaturized growth system of fast-growing plant material gives the throughput for a screening system. The plant material is grown in a cavity of a 96 well multi-well plate (e.g., ANSI Standard SLAS R2012) according to the following procedure: 150 μl of sterile plant growth media (3.9 g/l MS salts, 0.45 g/l MES, 10 g/l sucrose, ph 5.7 (with KOH) and 7 g/l agar) is transferred to each well of a 96 multi-well plate. Single surface sterilized seeds (3 h incubated in hydrochloric acid vapor) of the plant material are transferred to each cavity of a 96 multi-well plate. Multi-well plates are sealed with polythene foil and incubated for 2 days at 4° C. to break seed dormancy. After stratification, plates are transferred to plant growth chambers set to 16 h light (120 μmol m⁻² s⁻¹, 4000 K) and 8 h darkness and constant 22° C. After 5 days in the growth chambers, the plant material is treated with chemical substances.

### Example 2—Compound Application Procedure

The standardized application of chemical substances and the use of different concentrations results in a consistent analysis of plant material treatment effects of each individual chemical substance. In this embodiment, the plant material is grown according to the procedure as described in EXAMPLE 1. The plant material is treated with the chemical substances according to the following protocol: the chemical substances are each dissolved separately in 100% dimethyl sulfoxide (DMSO) as to provide a stock solution for each chemical substance. Stock solutions are diluted with water to a final DMSO concentration of 0.3%. 25 μl of the diluted solutions are applied on top of the solid growth media into each cavity of the multi-well plate by use of a pipette. In total, three different concentrations per chemical substance are applied separately to the plant material depending on the efficacy of each chemical substance. Applied concentrations range from 0.01 to 2000 g/ha. Chemical substances applied are listed in Table 1. Per cavity, only a single substance and solution (i.e., concentration) is applied.

In the description below, the following abbreviations stand for: ACCase: acetyl CoA carboxylase; ALS: acetolactate synthase; DXR: 1-deoxy-d-xylulose-5-phosphate reductoisomerase; DXS: 1-deoxy-D-xylulose 5-phosphate synthase; FAT: fatty acid thioesterases; HPPD: 4-hydroxyphenyl-pyruvate-dioxygenase; HST: homogentisate solanesyltransferase; PDS: phytoene desaturase; PPO: protoporphyrinogen oxidase; PSII: Photosystem II; and VLCFA: very long chain fatty acid synthesis.

Chemical substances listed in Table 1 have been applied on Arabidopsis thaliana plants to train a support vector machine model for SoA and/or MoA classification.

TABLE 1

| SoA/MoA | Chemical substance | CAS-number |
|---|---|---|
| ALS | Imazapyr | 81334-34-1 |
| ALS | Mesosulfuron-methyl | 208465-21-8 |
| ALS | Sulfosulfuron | 141776-32-1 |
| ALS | Cloransulam | 159518-97-5 |
| ALS | Foramsulfuron | 173159-57-4 |
| ALS | Thiencarbazone-methyl | 317815-83-1 |
| DXR/DXS | Fosmidomycin | 66508-53-0 |
| DXR/DXS | Ketoclomazone | 80959-17-7 |
| FAT | Oxaziclomefone | 153197-14-9 |
| FAT | Cinmethylin | 87818-31-3 |
| HPPD | Tembotrione | 335104-84-2 |
| HPPD | Sulcotrione | 99105-77-8 |
| HPPD | Mesotrione | 104206-82-8 |
| HPPD | Pyrazoxyfen | 71561-11-0 |
| HPPD | Pyrasulfotole | 365400-11-9 |
| HPPD | Tefuryltrione | 473278-76-1 |
| HPPD | Topramezone | 210631-68-8 |
| HPPD | Isoxaflutole | 141112-29-0 |
| Microtubule assembly | Oryzalin | 19044-88-3 |
| Microtubule assembly | Trifluralin | 1582-09-8 |
| Microtubule assembly | Tebutam | 35256-85-0 |
| Microtubule assembly | Benfluralin | 1861-40-1 |
| Microtubule assembly | Dinitramine | 29091-05-2 |
| PDS | Norflurazon | 27314-13-2 |
| PDS | Beflubutamid | 113614-08-7 |
| PDS | Fluridone | 59756-60-4 |
| PDS | Picolinafen | 137641-05-5 |
| PDS | Diflufenican | 83164-33-4 |
| PPO | Oxadiazon | 19666-30-9 |
| PPO | Lactofen | 77501-63-4 |
| PPO | Flumioxazin | 103361-09-7 |
| PPO | Acifluorfen | 50594-66-6 |
| PSII | Atrazine | 1912-24-9 |
| PSII | Isoproturon | 34123-59-6 |
| PSII | Diuron | 330-54-1 |
| PSII | Bromacil | 314-40-9 |
| PSII | Desmedipham | 13684-56-5 |
| PSII | Bentazone | 25057-89-0 |
| PSII | Metribuzin | 21087-64-9 |
| SPS | Aclonifen | 74070-46-5 |
| Synthetic auxins | 2,4-D | 94-75-7 |
| Synthetic auxins | Triclopyr | 55335-06-3 |
| Synthetic auxins | MCPA | 94-74-6 |
| Synthetic auxins | Dicamba | 1918-00-9 |
| Synthetic auxins | Benazolin-ethyl | 25059-80-7 |
| Synthetic auxins | 2,4-DB | 94-82-6 |
| Synthetic auxins | Clopyralid | 1702-17-6 |
| VLCFA | Flufenacet | 142459-58-3 |
| VLCFA | Piperophos | 24151-93-7 |
| VLCFA | Fentrazamide | 158237-07-1 |
| VLCFA | Pyroxasulfone | 447399-55-5 |
| VLCFA | Anilofos | 64249-01-0 |
| ACCase | Clethodim | 99129-21-2 |
| ACCase | Diclofop | 40843-25-2 |
| ACCase | Pinoxaden | 243973-20-8 |
| ACCase | Tralkoxydim | 87820-88-0 |
| EPSP synthase | Glyphosate | 1071-83-6 |
| Glutamine synthethase | Glufosinate | 35597-44-5 |
| Oxidant | Paraquat | 4685-14-7 |
| Cellulose synthesis | Dichlobenil | 1194-65-6 |
| Cellulose synthesis | Isoxaben | 82558-50-7 |

### Example 3—Procedure for Plant Imaging

The broad set of sensitive digital imaging systems applied hereunder ensures to detect effects of a treatment on plant material in necessary detail to assign a chemical substance used for the treatment to at least one SoA and/or MoA. In this embodiment, a dataset corresponding to phenotypical characteristics of the plant material is created according to the following procedure: Before treatment, as well as 24, 48, 72 and 96 h after treatment with the chemical substance, multi-well plates with the plant material in the single wells are taken out of the plant growth chambers and subsequently imaged with a hyperspectral camera VIS (e.g., GreenEye, InnoSpec GmbH, Germany), hyperspectral camera NIR (e.g., RedEye, InnoSpec GmbH, Germany), Pulse-Amplitude-Modulation chlorophyll fluorometer (e.g., Imaging PAM, WALZ GmbH, Germany) and a RGB camera (e.g., digital single lens reflex (SLR) camera—EOS700D, Canon AG, Japan). The GreenEye camera is set to a spectral range of 406 to 1190 nm, whereas the RedEye camera is set to 940 to 1743 nm. For both hyperspectral imaging cameras, an image of a white (polytetrafluorethylen) and dark reference (completely darkened measurement chamber or objective) is taken for calibration every day. Using white and dark references allows to express the measured reflectance per wavelength in relative, dimensionless unit scaled between 0 (signal dark reference) and 1 (signal white reference). The ImagingPAM is set to measure the quantum efficiency of photosystem II (overall photosynthetic capacity of the plant material) by measuring the chlorophyll fluorescence before and after a saturating light pulse (720 ms), emitted by a LED panel. With the EOS700D, a digital photograph is taken from each multi-well plate. After imaging of the plant material, multi-well plates are transferred back to the plant growth chambers.

### Example 4—Procedure for Image Analysis

An automated image analysis process allows to rapidly produce high quality datasets corresponding to phenotypical characteristics of the plant material and removes variation arising from the employed sensor units. The datasets are analyzed according to the following procedure: dark/white reference image pairs of the hyperspectral cameras are processed prior the handling of individual measurement images. The dark/white reference image pairs serve as a calibration system to address for differences in sensor sensitivity along spatial position and spectral dimension as well as differences in maximal positional reflectance due to light inhomogeneity and measurement day effects. Additionally, sensor artifacts (significant value drop along spatial dimension) and dead pixel spots (white-reference below dark-reference value) are corrected using linear interpolation between spatially neighboring pixels. The images are scaled based on the corresponding dark and white references for each spectral and spatial dimension. Smoothing along the spectral dimension using a Savitzky-Golay smoothing filter is performed to correct for signal noise. For cameras covering the visual wavelength range, synthetic RGB images are (re-)constructed using CIE 2006 color matching and D65 standard illuminant values. A predefined template of the multi-well plate is aligned using template matching and affine transformation based on cavity moments designated either based on initially detected plant material using Otsu's thresholding method (GreenEye) or from detected cavity border using Circle Hough transformation (RedEye). The aligned plant material masks facilitate a refined final plant material detection over designated image areas as well as the establishment of an unambiguous plant-material-to-cavity relation. The plant material is finally detected using Otsu's method over multiple selected spectral bands and image channels from reconstructed RGB image. For downstream statistical analysis and learning approach the detected plant material is summarized as mean and median spectral vector including deviation and exported in binary NetCDF portable data storage format. Data from the Imaging PAM is aligned to a predefined template of the multi-well plate using template matching and affine transformation with initially detected plant material from Otsu-based thresholding. Estimation of plant material size as well as quantum efficiency of plant photosystem II is based on common standard equations.

### Example 5—Soa/Moa Classification by Supervised Learning Program

Assigning a chemical substance to at least one SoA and or MoA is conducted according to the following process: The average reflectance spectrum and quantum efficiency of plant photosystem II data of all pixels from the plant material is used for classification. The spectral range of 446.8 to 1127.8 nm with 178 spectral bands is considered for the analysis of the hyperspectral images. All plant material pieces with a reflectance above 0.35 units at 677 nm at the images taken before application of the compound are removed to filter plant materials which are too small or tilted from the view plane. Furthermore, all plant material pieces with a reflectance below 0.01 units at 677 nm at any time point are discarded to eliminate ungerminated plant material or molded cavities of the multi-well plate. To identify plant material pieces, which were not treated or were not growing properly in the measured time scale, an outlier detection is performed for each treatment and concentration at 96 h after treatment by using the automated measured size of the plant material. To reduce the influence of the plant material size, values are rescaled from 0 to 1 (same range as the other data types). A z-score for each plant material over the spectral bands is calculated to normalize for small shifts between plant materials from different cavities of a multi-well plate. Data from the plant material pieces which were treated with the highest chemical substance concentration are considered for analysis. Hyperspectral imaging data, as well as the size obtained from plant material pieces 96 h after application of the chemical substance and the quantum efficiency of photosystem II of plant material pieces obtained 24 h after application are used for classification. For datasets of chemical substances with a specific SoA and/or MoA, a sampling with replacement is performed to balance the number of measurements for each SoA and/or MoA. On average, 80 individual measurements at each measured time point and applied concentration of different plant materials are used per chemical substance for the classification. A multi-class support vector machine (SVM) is trained on a compendium corresponding to datasets of plant material treated with chemical substances listed in Table 1 to classify the SoA and/or MoA of each treated plant material. Finally, a majority vote of the obtained classification of the on average 80 individual plant material pieces of the classified SoA and/or MoA classes for all individually treated plant material pieces of a single chemical substance are then used to classify the SoA and/or MoA of a specific chemical substance.

Further advantages, advantageous embodiments and developments of the invention will become apparent from the exemplary embodiments, which are described below in association with the schematic figures.

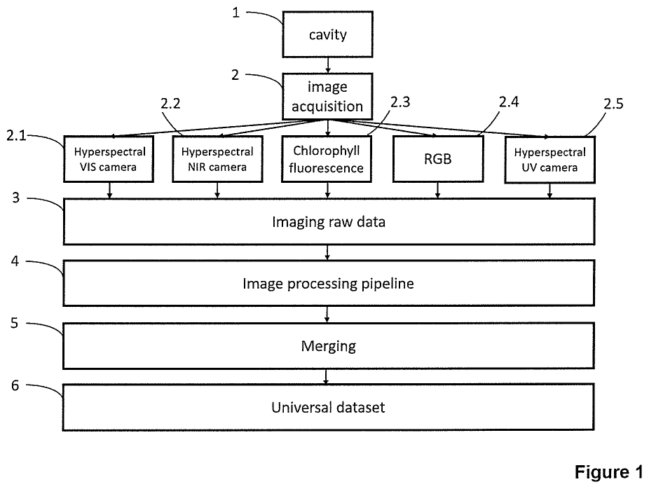

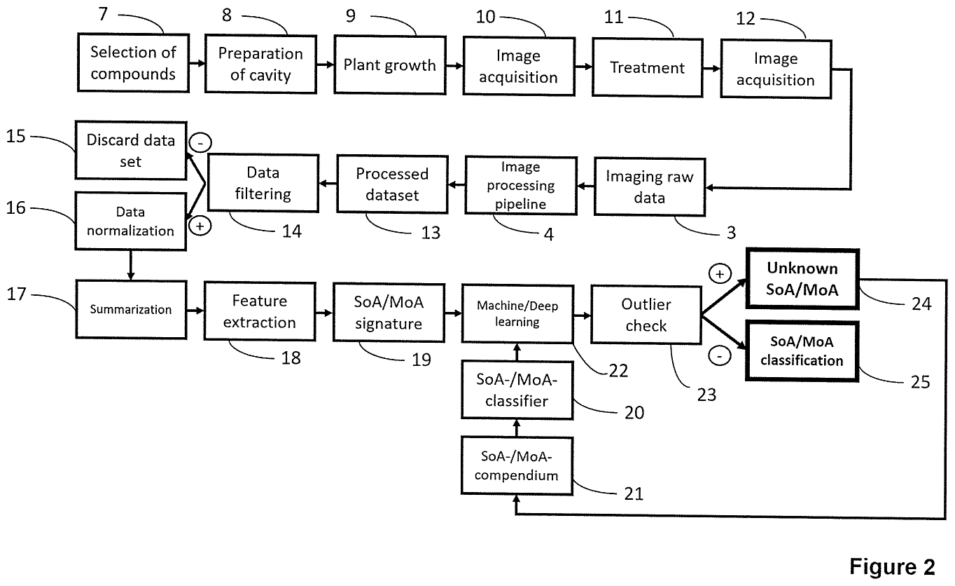

FIG. 1 shows an overview of different stages of data acquisition in the method according to the invention;

FIG. 2 shows an overview of the method according to the invention;

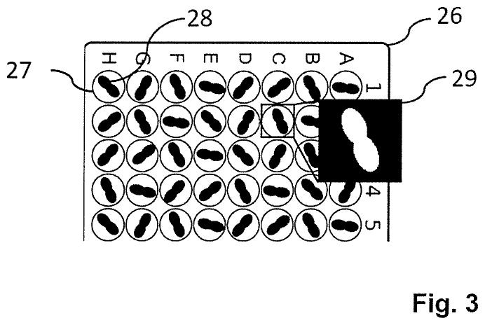

FIG. 3 shows an exemplary cutout of a multi-well plate used for the method according to the invention;

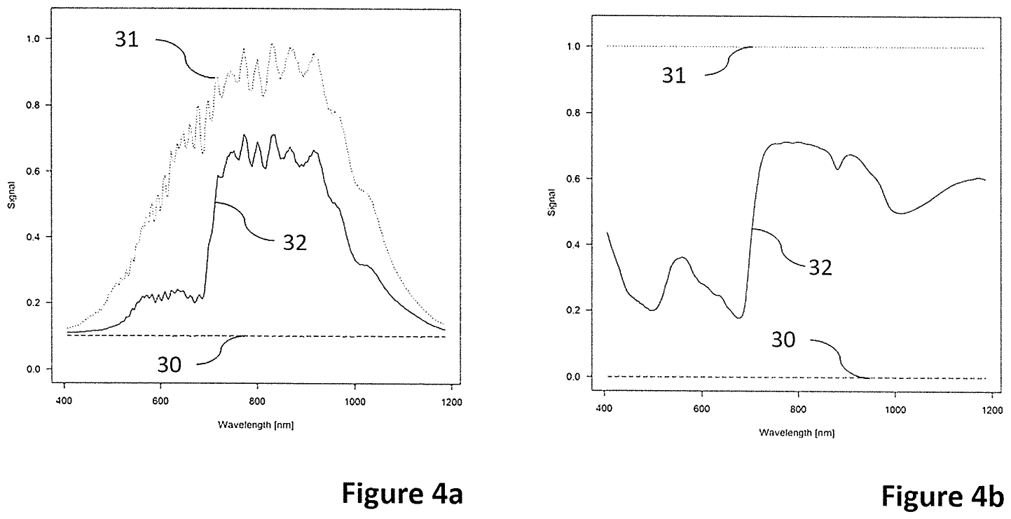

FIG. 4a shows an exemplary plot displaying raw data used in the method according to the invention;

FIG. 4b shows an exemplary plot displaying data of FIG. 4a in a scaled and smoothed way;

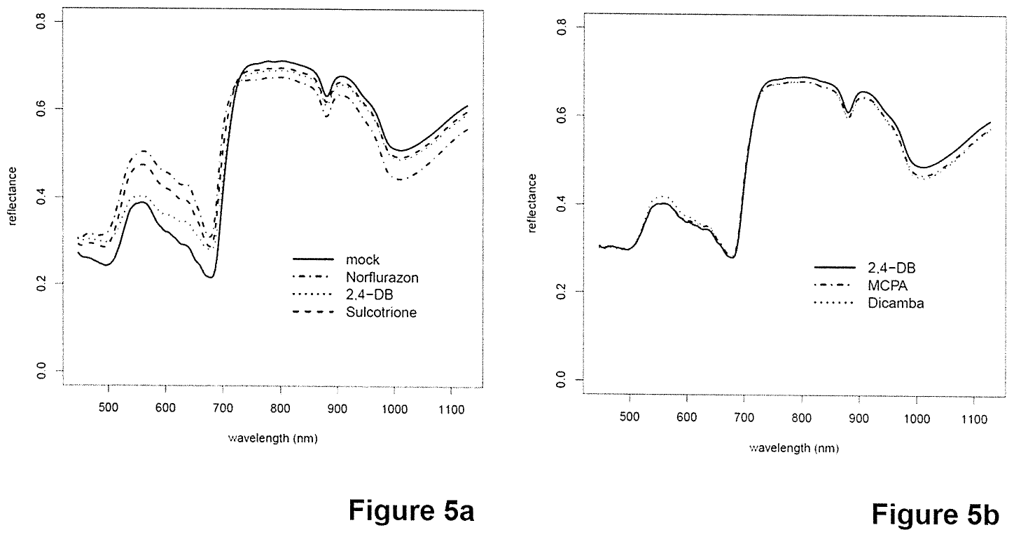

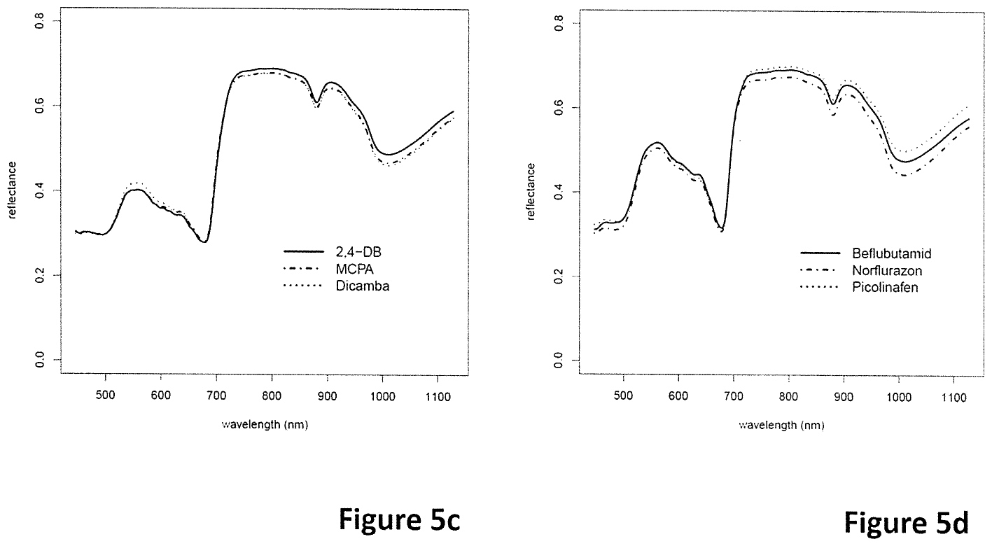

FIG. 5a-d show mean spectra of plant material treated with different known chemical substances obtained in the method according to the invention;

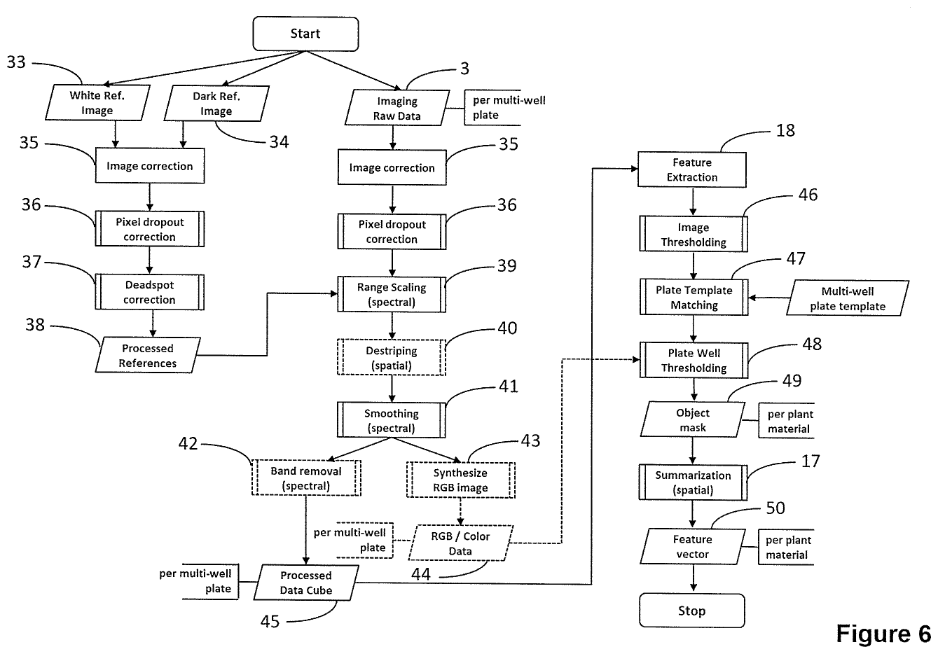

FIG. 6 shows an exemplary code workflow for image processing using hyperspectral imaging for obtaining datasets;

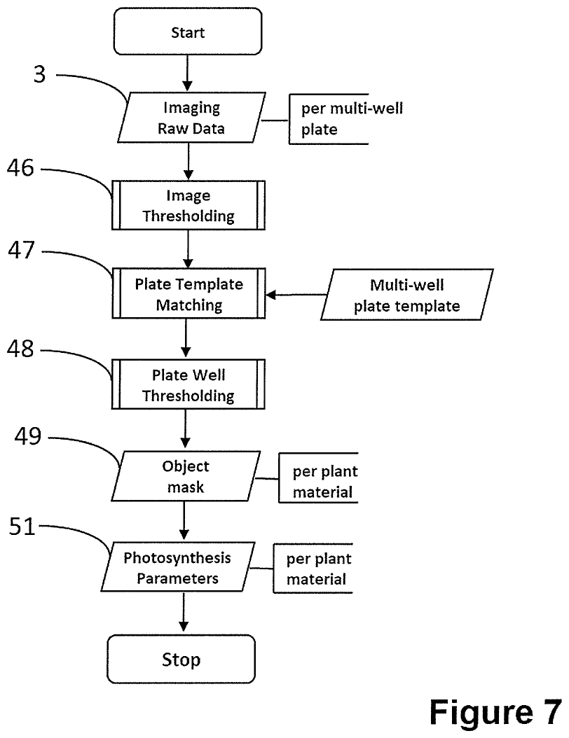

FIG. 7 shows an exemplary code workflow for image processing using ImagingPAM for obtaining datasets; and

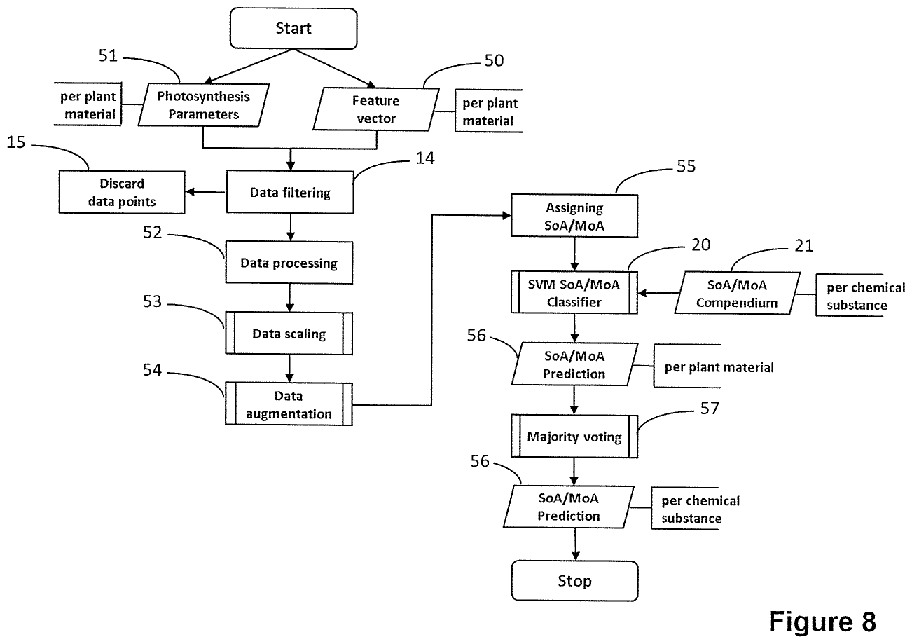

FIG. 8 shows an exemplary code workflow for SoA/MoA classification.

FIG. 1 shows an overview of different stages of data acquisition in the method according to the invention. In step 1, plant material is applied into a cavity, for example a well of a multi-well plate. Afterwards, data is obtained in step 2 of the method. In this embodiment, datasets of the plant material are taken via digital optical sensors. Data acquisition may be carried out by one or several different sensors, as for example by a hyperspectral VIS sensor 2.1, a hyperspectral NIR sensor 2.2, a chlorophyll fluorescence sensor 2.3, an RGB sensor 2.4 or a hyperspectral UV sensor 2.5. A combination of different sensors of step 2 is advantageous, for instance data acquisition by use of the hyperspectral VIS sensor 2.1, the hyperspectral NIR sensor 2.2 and the chlorophyll fluorescence sensor 2.3. The use of these sensors is preferred, whereby the RGB sensor 2.4 and the hyperspectral UV sensor 2.5 may also be used optionally. In step 3, the raw data sets taken in step 2 are collected. The image processing pipeline 4 acts as a feature extractor representing all obtained data for plant material (different sensors/spatial and spectral information) in a feature vector. All datasets are merged in step 5. At the end of the method, a universal dataset is gathered (step 6).

FIG. 2 shows an overview of the method according to the invention. At the beginning, a selection of compounds 7 takes place. In this step, one or more chemical substances to be screened, plant material and data acquisition sensors are selected. The chemical substances can be manually selected covering established herbicidal SoA and/or MoA or uncharacterized ones. In step 8, one cavity per piece of plant material is prepared. Each cavity is equipped with growth medium for the plant material. In this specific example, Arabidopsis thaliana seeds are used as plant material. One seed is put into one cavity. This ensures that phenotypic data and derived parameters of one plant can be extracted by the image analysis pipeline. The cavity in this example is a well of a multi-well plate, as the use of multi-well plates facilitates handling. Before the multi-well plate is put into a growth chamber for plant growth 9, stratification is carried out to break seed dormancy. Standard growth conditions in this embodiment include 5 days of pre-growth on solid growth media with sucrose in a growth chamber with 16 h light per day (120 μmol m⁻² s⁻¹) and 8 h of darkness. Temperature inside the growth chamber is constantly kept at 22° C. Different growth conditions can be adapted in the screening method to enhance specific SoA and/or MoA, which affect plant materials marginal or not at all under standard growth conditions. Growth conditions can also be adapted for different plant species. After the pre-growth, a first dataset of the plant material is obtained just before treatment 11. In this embodiment, this is carried out via image acquisition (pre-treatment) 10 with different sensors, preferably by use of a combination of the hyperspectral VIS sensor, the hyperspectral NIR sensor and the chlorophyll fluorescence sensor. The use of these sensors enables the recording of digital imaging data with a broad spectrum and high resolution to characterize the phenotype of the plant material. The combination of these sensors with customized acquisition software for full parameter control and automation of image acquisition and processing is preferred. One collective dataset per sensor is taken of the whole multi-well plate. Afterwards, treatment 11 can be carried out by pipetting or foliar application via spraying of an aerosol. Each multi-well plate contains control wells with untreated plant materials. This helps to identify process errors. In this embodiment, image acquisition (after treatment) 12 is carried out 24, 48, 72 and 96 h after treatment 11. In the next step (imaging raw data 3) the dataset taken for the multi-well plate is divided into single datasets for each single well containing one seedling, further analyzed by an image processing pipeline 4 and merged to a processed dataset 13. Data filtering 14 helps to identify ungerminated seeds, contaminated wells or plant material that has not grown optimal before treatment. These datasets are excluded from further analysis in step 15. Data normalization 16 is carried out by a mixed-effects model for phenotypic data of the treated plant material to remove confounding effects. This step is crucial to compare data over time (for weeks, months and years). After data normalization 16, datasets are summarized in step 17 for further processing. The automated feature extraction 18 serves to extract the most relevant phenotypic characteristics. SoA and/or MoA signatures gathered in the previous steps are assigned to the data in step 19. An algorithm is trained subsequently on recognition of SoA and/or MoA. After training it, the model is validated. In case of non-validated data, the algorithm has to be trained further and the data is reviewed again. The use of chemical substances with known SoA and/or MoA is essential for the proper training and validation of the algorithm. When the data is validated, a SoA- and/or MoA-classifier 20 is trained based on this data consisting of SoA and/or MoA of known chemical substances. The data obtained in the training phase is used to build this mathematical classification model trained to predict the correct SoA and/or MoA of the chemical substance by supervised machine learning (including for example random forest or support vector machines). Classical machine as well as deep learning is used on all data points to accurately classify SoA and/or MoA. Data assigned by the SoA- and/or MoA-classifier 20 is then stored in a SoA- and/MoA-compendium 21. The outlier check 23 is used to categorize the SoA and/or MoA: if the data matches data patterns stored in the SoA- and/MoA-compendium 21, classification 25 takes place. In case the data does not match any of the stored data, an uncharacterized (potential novel) SoA and/or MoA is detected (step 24). The detection of an uncharacterized MoA and/or SoA requires further tasks and opens up the possibility of adding further data to the MoA- and/or SoA-compendium.

FIG. 3 shows a cutout of a multi-well plate 26 comprising 96 cavities 27, which are automatically detected by an image handling process. Each cavity 27 contains one plant material 28. In further analysis the pixels associated with plant material 28 can be separated from a background 29 pictured in a zoom-in of a single cavity 27. This way the background comprising growth media and/or the cavity can be excluded from further data processing.

In FIGS. 4a and 4b, two different exemplary plots of spectral imaging data are shown: FIG. 4a displays raw data, while FIG. 4b shows scaled and smoothed data. The sensitivity of hyperspectral camera sensors is different depending on the spectral range. To normalize and scale this effect, it is necessary to have white and dark references. In both plots, FIG. 4a and FIG. 4b, the dash line 30 represents a dark reference, whereas the dotted line 31 displays the white reference (imaging of highly reflecting material: Polytetrafluorethylen) and the solid line 32 the plant material. On the outer regions of a here shown VIS spectrum the sensitivity is lower than in the center (illustrated in FIG. 4a) and the plant material data is between the white and dark references. The raw data displayed in FIG. 4a is edited in the image processing pipeline. FIG. 4b shows the scaled and smoothed data. The scaling ensures to consider the dynamic range over the whole spectra whereas the smoothing of the data planish small fluctuations and therefore strengthen relevant changes in the spectra.

FIG. 5a-d show mean spectra of plant material (Arabidopsis thaliana) treated with different known chemical substances to illustrate the effect of the treatment on the spectra of the plant material. The different spectra each represent the mean value of datasets obtained from a multitude of plant material 96 h after application of the chemical substances. Applied concentrations per chemical substance are mentioned in brackets: FIG. 5a shows the spectra of a control treatment (mock) and three treatments with different SoA/MoA: Norflurazone (5 g/ha)—phytoene desaturase (PDS) inhibitor; 2,4-DB (100 g/ha)—synthetic Auxins; and Sulcotrione (2 g/ha)—4-hydroxyphenyl-pyruvate-dioxygenase (HPPD) inhibitor. These spectra exemplify the effect of treatments with different SoA and/or MoA, which add up to different spectra of the plant material. FIGS. 5b-d show spectra of plant material treated with chemical substances which act by the same SoA and/or MoA. Plant material of these spectra cluster together. The results shown in FIG. 5b are obtained by use of synthetic Auxins: 2,4-DB (100 g/ha); MCBA (10 g/ha); and Dicamba (50 g/ha). The results shown in FIG. 5c are obtained by use of phytoene desaturase (PDS) inhibitors: Beflubutamid (10 g/ha); Norflurazon (5 g/ha); and Picolinafen (1 g/ha). Results shown in FIG. 5d are obtained by use of 4-hydroxyphenyl-pyruvate-dioxygenase (HPPD) inhibitors: Sulcotrione (2 g/ha); Mesotrione (1 g/ha); and Topramezone (100 g/ha). For the results shown in FIGS. 5a-d, Arabidopsis thaliana (accession Col-0) plants are grown as described in EXAMPLE 1. The chemical substances are applied as described in EXAMPLE 2. 96 plants per treatment and concentration are analyzed. Image acquisition is carried out before the treatment as well as 24, 48, 72 and 96 h after the treatment as described in EXAMPLE 3. The data created is processed as described in EXAMPLE 4 and datasets are generated for SoA and/or MoA classification. Assigning of chemical substances with known SoA and or MoA is conducted as described in EXAMPLE 5 with each of the chemical substances listed in table 1 used as test data. On average, the phenotypical characteristics of plant materials treated with five chemical substances describe one SoA and/or MoA. Classification performance is tested by preferably leaving each chemical substance out of the training dataset and testing the assignment of individual plant materials to the correct SoA and/or MoA (table 2a). For SoA and/or MoA with less than two chemical substances available, accuracy is calculated based on classical cross validation (table 2b). Classification performance is evaluated using the prediction accuracy as a statistical measure of how well a classification model correctly identifies the correct MoA and/or SoA. Here, accuracy is the proportion of correct predictions (both true positives and true negatives) among the total number of cases tested.

TABLE 2a

| SoA/MoA | Accuracy |
|---|---|
| ACCase | 0.791176 |
| ALS | 0.770563 |
| DXR/DXS | 0.454936 |
| FAT | 0.458333 |
| HPPD | 0.864198 |
| Microtubule assembly | 0.701847 |
| PDS | 0.682927 |
| PPO | 0.705989 |
| PSII | 0.932347 |
| Synthetic auxins | 0.60452 |
| VLCFA | 0.721925 |

TABLE 2b

| SoA/MoA | Accuracy |
|---|---|
| ACCase | 0.842324 |
| ALS | 0.92579 |
| Cellulose biosynthesis | 0.735509 |
| DXR/DXS | 0.688025 |
| EPSP synthase | 0.429589 |
| FAT | 0.468932 |
| Glutamine synthethase | 0.885473 |
| HPPD | 0.92269 |
| Microtubule assembly | 0.83791 |
| PDS | 0.839713 |
| PPO | 0.845077 |
| PSII | 0.954997 |
| SPS | 0.648352 |
| Synthetic auxins | 0.839989 |
| VLCFA | 0.796927 |

FIG. 6 shows an exemplary code workflow for image processing using hyperspectral imaging to obtain datasets. In step 3, raw data imaging is carried out for one multi-well plate at once. Each dataset obtained may later be divided into single datasets per piece of plant material, but at this point of the workflow, one dataset per multi-well plate is used. Image correction 35 and pixel dropout correction 36 takes place for the dataset as well as for white and dark reference images 33,34 used for normalization. The white and dark reference images 33,34 undergo deadspot correction 37 before building a base of processed references 38 that can later be used for spectral range scaling 39. Spatial destriping 40 may optionally be carried out before spectral smoothing 41, which is leading to step 42, where optionally spectral band removal is conducted to avoid lower accuracy of the later-built classifier. The processed data cube 45 is then used for feature extraction 18. Step 46 describes an image thresholding process wherein together with step 47, the plate template matching, the data points of the dataset are categorized as background or foreground data. The plate template matching 47 is still carried out on multi-well plate template level. Afterwards, the thresholding is repeated in step 48 on plate well level. If the optional step 43, the synthesizing of an RGB image, was previously carried out, the RGB/color data 44 that is obtained on multi-well plate level, can be used at this point as well. To acquire datasets per single piece of plant material, object masking 49 is conducted. In this step, data belonging to each single piece of plant material in the multi-well plate is masked out individually creating multiple single datasets. After spatial summarization 17, a feature vector 50 per single piece of plant material is consecutively built before stopping the workflow shown in FIG. 6.

FIG. 7 shows an exemplary code workflow for image processing using ImagingPAM for obtaining datasets. In a first step, the imaging of the raw data 3 per multi-well plate and afterwards image thresholding 46 is carried out. The background-corrected dataset is then compared to a multi-well plate template for plate template matching 47 before the thresholding takes places for the plate wells in step 48. As previously shown in FIG. 6, the single pieces of plant material are masked out individually in step 49. In the last step before stopping the workflow shown in FIG. 7, photosynthesis parameters 51 for each dataset of a single piece of plant material are determined based on the data processed.

FIG. 8 shows a code workflow for SoA and/or MoA classification. After starting the workflow, photosynthesis parameters 51 and the feature vector 50, both per single piece of plant material, are combined. Afterwards, data filtering 14 is conducted. If the data is not sufficient, these data points are discarded in step 17. Data passing the filtering 14 is processed in step 52, by scaling the dataset in step 53 and augmented in step 54. The dataset created this way is then used for the assigning of phenotypical characteristics of the plant material to one or more SoA and/or MoA (step 55). A support vector machine (SVM) SoA- and/or MoA-classifier 20 based on the SoA- and/or MoA-compendium 21 is used for the SoA and/or MoA prediction 56 per single piece of plant material. By majority voting 57, SoA and/or MoA prediction 56 is carried out per chemical substance before stopping the workflow exemplified in FIG. 8.

1. A method for screening of at least one chemical substance by treatment of plant material, comprising the steps of:
a. applying the plant material into a cavity;
b. treatment of the plant material with the chemical substance;
c. creating at least one dataset showing at least one phenotypical characteristic of the plant material after treatment with the chemical substance; and
d. assigning the chemical substance based on the dataset to at least one site of action (SoA) and/or at least one mode of action (MoA) of a multitude of stored SoA and/or MoA by using a SoA- and/or MoA-compendium containing data regarding dependencies between phenotypical characteristics of at least one plant material treated by at least one reference substance of a known SoA and/or MoA.

2. The method according to claim 1, wherein the dataset is obtained by use of a sensor unit.

3. The method according to claim 2, wherein the sensor unit is used to obtain the dataset in a non-destructive and non-intrusive matter.

4. The method according to claim 2, wherein the sensor unit comprises at least one of hyperspectral VIS, hyperspectral NIR, chlorophyll fluorescence or RGB sensors.

5. The method according to claim 4, wherein a light source with a circular arrangement of lamps and reflective surface is used for the hyperspectral VIS and hyperspectral NIR sensors to homogeneously illuminate the plant material.

6. The method according to claim 1, wherein the cavity is a well of a multi-well plate.

7. The method according to claim 6, wherein each well contains one piece of plant material.

8. The method according to claim 1, wherein the chemical substance is applied at different concentrations.

9. The method according to claim 1, wherein one collective dataset is taken for a multitude of plant material pieces and subsequently decomposed into single datasets per single piece of plant material.

10. The method according to claim 4, wherein multiple datasets of the plant material are obtained after treatment at different predefined times or by the use of different sensors.

11. The method according to claim 1, wherein the dataset of the plant material can subsequently be decomposed into single datasets of different parts of the plant material.

12. The method according to claim 1, wherein at least one dataset showing at least one phenotypical characteristic of the plant material before treatment with the chemical substance is obtained.

13. The method according to claim 1, wherein the assigning of the chemical substance to at least one SoA or at least one MoA is carried out by using an adapted program performing a machine learning process.

14. The ethod according to claim 1, wherein the SoA- and/or MoA-compendium is augmented by recording of data of at least one reference substance of a further SoA and/or MoA.

15. The method according to claim 1, wherein an uncharacterized SoA and/or MoA is identified for each chemical substance not assignable to any recorded SoA and/or MoA in the SoA- and/or MoA-compendium.

16. The method according to claim 1, wherein the plant material is in a seed or seedling stage at step a.

17. The method according to claim 1, wherein the plant material belongs to the plant species Arabidopsis thaliana.

18. The method according to claim 1, wherein the chemical substance is a plant growth regulator.
